(19) United States
(12) Patent Application Publication
(10) Pub. No.: US 2011/0274238 A1
(43) Pub. Date: Nov. 10, 2011

(54) Computed tomography system

(76) Inventor: Michael MASCHKE, Lonnerstadt (DE)

(21) Appl. No.: 13/103,160

(22) Filed: May 9, 2011

(30) Foreign Application Priority Data
May 10, 2010 (DE) 10 2010 019 991.5

(51) Int. Cl. A61B 6/03; B82Y 5/00

(52) U.S. Cl. 378/9; 378/4; 378/16; 378/19; 977/742; 977/904; 977/950

(57) ABSTRACT

A computed tomography system is provided for allowing it to be deployed more effectively. The computed tomography system features an annular CT gantry with a central opening, a recording system that can be rotated within the gantry. The recording system has an x-ray source and an x-ray detector apparatus. It is possible to remove a part of the ring of the CT gantry from the ring at least partially so that there is a break in the ring, through which an examination object can be moved into the central opening. The computed tomography system is for phase contrast x-ray imaging.

### CROSS REFERENCE TO RELATED APPLICATIONS

This application claims priority of German application No. 10 2010 019 991.5 filed May 10, 2010, which is incorporated by reference herein in its entirety.

### FIELD OF THE INVENTION

The invention relates to a computed tomography system.

### BACKGROUND OF THE INVENTION

Projection x-ray recording is one of the medical examinations used most frequently for imaging purposes. Typical recordings include thoracic recordings and pelvic recordings on a standing patient, cranial recordings, recordings of the arms or legs to ascertain a break in such limbs for example. In this process an x-ray source emits x-ray beams onto an examination object; these penetrate the examination object, some of them being reflected or absorbed in the tissue, producing an x-ray image on an x-ray detector located behind the examination object. A vacuum tube is used here to produce ionizing radiation. The x-ray image produced is based on the differing rates of absorption of x-rays in different materials, e.g. bone compared with soft tissue. For examinations which require a particularly good representation of soft tissue the projection x-ray recording is replaced by computed tomography recording, which produces three-dimensional x-ray images. One disadvantage of computed tomography is the high overall body dose to which the patient is exposed.

In recent years there has been increasing research in the field of x-ray-based phase contrast. Phase contrast x-ray imaging achieves a much better contrast in certain applications, in particular with soft tissue. The underlying technology for this is already known from electron microscopy. The most recent research results now allow phase contrast x-ray imaging to be achieved with a conventional x-ray source with the aid of three aligned gratings, a coherence grating, a phase grating and an amplitude grating, utilizing the Talbot effect. The technology is known for example from EP 1 879 020 A1; a phase contrast CT is known from US 2007/0183560 A1.

### SUMMARY OF THE INVENTION

The object of the present invention is to provide a computed tomography system that ensures particularly good image quality with the lowest possible x-ray exposure and a versatile structure.

The object is achieved by a computed tomography system according to the independent claim. Advantageous embodiments of the invention are respectively the subject matter of the associated dependent claims.

The inventive computed tomography system, featuring an annular CT gantry with a central opening, a recording system that can be rotated within the gantry having an x-ray source and an x-ray detector apparatus, it being possible to remove a part of the ring of the CT gantry from the ring at least partially so that there is a break in the ring, through which an examination object can be moved into the central opening, the computed tomography system being configured for phase contrast x-ray imaging, offers a particularly good image quality in particular for soft tissue representation with a relatively low x-ray dose for an examination object. This allows more precise diagnoses to be made and patient exposure is low. The break in the gantry allows the computed tomography system to be used in a particularly flexible manner, since for example it is particularly easy to position the examination object and the examination object or patient can be accessed particularly easily by a physician.

According to one embodiment of the invention the CT has a phase grating, which is disposed between an examination object and the x-ray detector apparatus, and an amplitude grating, which is disposed between the phase grating and the x-ray detector apparatus.

The fundamental idea of phase contrast x-ray imaging is to locate the precise positions of interference lines produced by means of the phase grating from coherent x-ray radiation passing through an examination object and to use these to determine the phase displacement through the examination object. However since the distances between the interference lines are in the micrometer range, a standard x-ray detector does not have adequate resolution to map the interference lines or their maxima. For this reason an absorption grating or amplitude grating with low x-ray transparency and where possible the same periodicity and orientation as the interference lines is disposed directly in front of the x-ray detector and used to sample the interference lines.

The phase grating or diffraction grating therefore produces an interference pattern, which maps a moiré pattern on the x-ray detector behind with the aid of the absorption grating or amplitude grating. If the absorption grating is displaced slightly, the moiré pattern is likewise displaced, bringing about a change in the local intensity in the x-ray detector behind, which can be determined relative to the displacement of the absorption grating. If the intensity change as a function of the displacement path of the absorption grating is applied for each detector element of said grating, in other words for every beam, it is possible to determine the phase displacement of the respective beam. The absorption grating or amplitude grating therefore performs the function of a transmission mask and converts local interference lines to intensity fluctuations. The measured signal profile contains quantitative information about the phase gradient of the examination object.

The CT advantageously has means for moving the amplitude grating perpendicular to the radiation direction of the x-ray radiation.

The diffraction grating or phase grating is structured two-dimensionally for example and has a low x-ray absorption. At the same time it produces a clear phase displacement, for example of π or an odd multiple thereof. The diffraction grating or phase grating can be made of silicon or a polymer for example. It can also be configured as a beam splitter grating.

The absorption grating or amplitude grating is likewise structured two-dimensionally and has a high x-ray absorption. It is disposed directly in front of the x-ray detector in the radiation direction for example and performs the function of a noise suppression grating.

According to a further embodiment of the invention the x-ray source features a plurality of field emission x-ray sources for emitting quasi-coherent x-ray radiation. Integrating an x-ray emitter with field emission x-ray sources means that there is no need for the complex source grating to produce monochromatic x-ray radiation, since the field emission x-ray sources are a simple and outlay-free option for producing quasi-coherent x-rays with a narrow focus. This allows the CT to be produced in a particularly compact and low-cost manner.

The field emission x-ray sources can expediently be disposed in an array, in other words for example in a two-dimensional, matrix-type arrangement. Because of their small size the field emission x-ray sources can be disposed very close to one another, so that a surface is formed from x-ray focal points.

With a field emission x-ray source or the associated field emission cathode electrons are emitted by applying a sufficiently large electric field. Field emission is achieved for example by a simple diode mode, in which a prevoltage is applied between anode and cathode. Electrons are emitted by the cathode when the electric field exceeds the threshold for emission. A triode construction can also be provided, in which a gate electrode is disposed close to the cathode. Electrons are emitted here by applying a prevoltage between gate and cathode. The emitted electrons are then accelerated by a high voltage between gate and anode. Field emission cathodes allow a very high, easily controlled and easily focused electron beam stream. Generally the field emission emitter and the field emission x-ray sources mean that the invention has the advantages of low heat generation at the x-ray source and a low weight, both due to the field emission emitter itself and also due to the lack of need for or reduction of a cooling system. Such a field emission emitter also has a high level of compactness compared with conventional x-ray emitters, with the result that a high-quality, flat x-ray source with a surface of many adjacently disposed focal points is possible for the first time. This is ensured in particular by an array with a plurality of field emission emitters. Also the service life of field emission emitters is much longer than that of known x-ray emitters with thermal cathodes. Also a field emission cathode can be started up quickly without heating unlike a thermal cathode. Additionally the easily focused electron stream means that a higher spatial resolution can be achieved for x-ray imaging.

According to one embodiment of the invention a field emission x-ray source features a field emission cathode in each instance with a nanostructured material with carbon nano tubes, known as a CNT or carbon nano tube cathode. Such materials have a particularly good emission characteristic, are stable even at high currents and can also be produced in a particularly small size. Field emission technology using CNT is known for example from U.S. Pat. No. 7,359,484 B2.

Alternatively a conventional x-ray source can also be used. The CT then has a further grating, which is disposed behind the x-ray source and is configured to produce quasi-coherent x-ray radiation from the x-ray radiation of the x-ray source.

According to a further embodiment of the invention the gantry features at least two recording systems, each with one x-ray source and one x-ray detector apparatus. In a CT with two recording systems the first recording system can advantageously be configured for phase contrast x-ray imaging and the second recording system for fluoroscopy x-ray imaging. This allows the CT both to provide conventional recordings and also to supply particularly high-quality soft tissue representations, making it also suitable for minimally invasive interventions and minimally surgical treatments.

Advantageously for particularly efficient, fast and simple spatial adjustment the CT gantry is disposed in a pivotable manner on a mount, in particular a ceiling mount.

According to a further embodiment of the invention the part is configured so that it can be pivoted out from the ring of the gantry. The part is preferably a ring segment, which is disposed on the remainder of the ring by means of hinges for example and can be pivoted away as required. Alternatively the ring segment can also be pushed into the remainder of the ring, so that there is a break. According to a further alternative the partial segment can also be decoupled completely from the CT gantry.

### BRIEF DESCRIPTION OF THE DRAWINGS

The invention and further advantageous embodiments according to the features of the subclaims are described in more detail below with reference to schematically illustrated exemplary embodiments in the drawing without in so doing restricting the invention to these exemplary embodiments. In the drawing:

FIG. 1 shows a structure of an inventive computed tomography system for phase contrast x-ray imaging with closed gantry,

FIG. 2 shows a structure of an inventive computed tomography system for phase contrast x-ray imaging with opened gantry,

FIG. 3 shows a structure of a beam profile during phase contrast x-ray imaging with field emission x-ray sources,

FIG. 4 shows a structure of a gantry of an inventive CT,

FIG. 5 shows a view of a field emission emitter with a number of field emission cathodes,

FIG. 6 shows a structure of an x-ray recording system for phase contrast x-ray imaging according to the prior art,

FIG. 7 shows a diagram of the radiation profile and the phase gradient during phase contrast x-ray imaging without examination object according to the prior art and

FIG. 8 shows a diagram of the radiation profile and the phase gradient during phase contrast x-ray imaging with examination object according to the prior art.

### DETAILED DESCRIPTION OF THE INVENTION

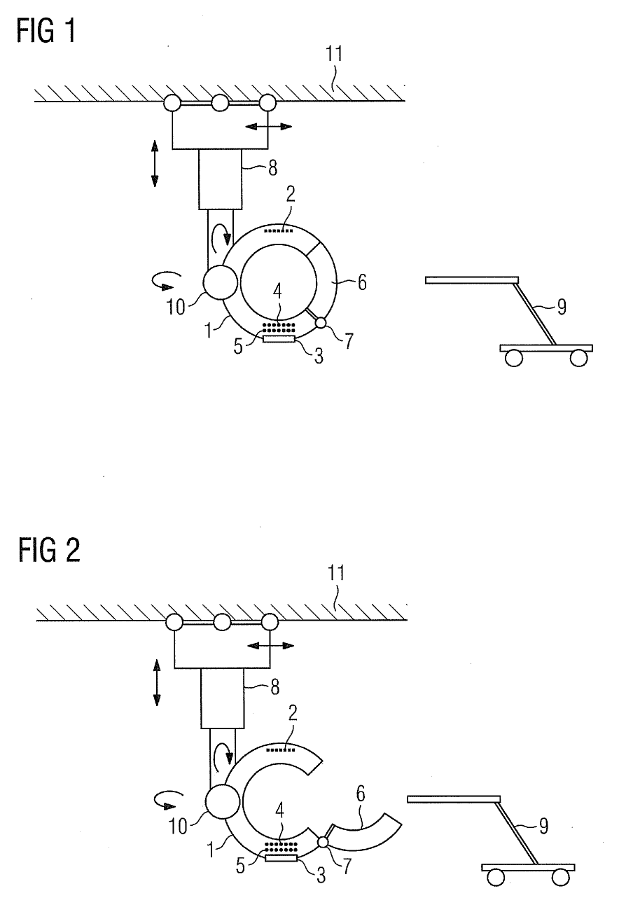

FIG. 1 shows an inventive computed tomography system with a CT gantry 1, the CT gantry 1 having a ring segment 6, the position of which can be changed relative to the remainder of the gantry. The gantry 1 is configured as annular and features a central opening 17 in which an examination object can be positioned. The gantry 1 also features a hollow space in the interior of the ring, in which a recording system is disposed in such a manner that x-ray images can be recorded of the examination object disposed in the opening. The recording system can rotate about the opening (through 360°) within the hollow space and can record x-ray images as it rotates. The recording system is configured for phase contrast x-ray imaging and in addition to an x-ray source in the form of a field emission emitter 2 and an x-ray detector apparatus 3 it also features a phase grating 4 and an amplitude grating 5. The field emission emitter 2 features a plurality of field emission x-ray sources 12, e.g. in that a field emission emitter with a plurality of field emission cathodes—as shown in FIG. 5—is provided. A number of field emission emitters with one field emission cathode each can also be provided.

The field emission x-ray sources are disposed for example in an array, which is structured in the manner of a matrix. The field emission x-ray sources can also be disposed in rows or columns. The field emission x-ray sources can optionally be activated individually, in rows or columns or as an entire matrix for emitting x-ray radiation. The field emission emitter 2 produces quasi-coherent x-ray radiation. The phase grating 4 is disposed behind the examination object between the x-ray source and the x-ray detector apparatus 3, the amplitude grating 5 between the phase grating 4 and the x-ray detector apparatus 3.

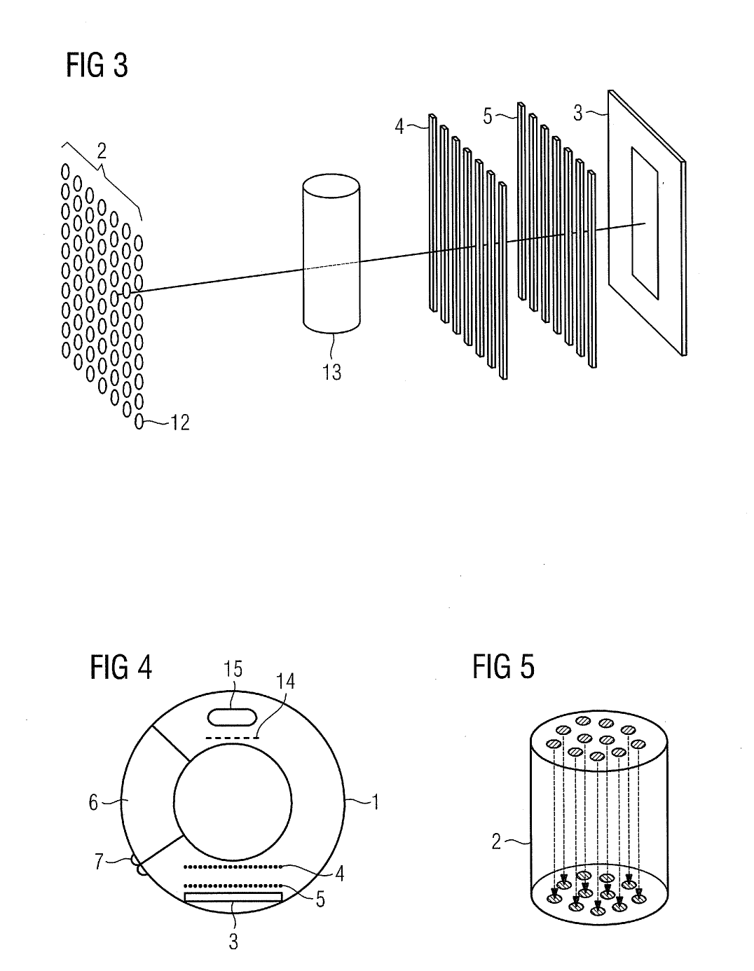

Phase contrast x-ray imaging utilizes the fact that different body tissue interrupts x-rays to differing degrees. The basic structure during phase contrast x-ray imaging with field emission cathodes producing coherent radiation is shown again in FIG. 3. The coherent x-ray radiation penetrates an examination object 13. The grating diffracts the coherent radiation in the known manner, producing an interference pattern and converting this correspondingly to intensity fluctuations. The measured signal profile then contains quantitative information about the phase gradients of the examination object. The amplitude grating 5 here is advantageously moved perpendicular to the radiation direction of the x-ray radiation, for example by a motor or a piezo actuator.

The ring segment 6 of the gantry 1 can be folded out of the ring of the gantry 1, as shown in FIG. 2, so that the ring of the gantry 1 is broken. In a first position of the ring segment (FIG. 1) the ring segment together with the remainder of the gantry forms a continuous ring, in which the recording system can also be rotated. In a second position (FIG. 2) the ring segment 6 is folded out of the gantry, the ring segment 6 being separated on one side and folded away from the other side by means of a hinge 7.

Alternatively the ring segment 6 can also be removed completely from the gantry 1, in that both sides are configured so that they can be separated. In another alternative it may be possible to push the ring segment 6 into the remainder of the gantry 1, so that a break likewise results. The break for example allows an examination object disposed on a patient couch 9 to be introduced into the central opening of the gantry. The ring segment is then returned to its first position and x-ray images can be recorded. The movements of the ring segment can be brought about by means of an external power drive for example. A CT with a detachable ring segment is known for example from U.S. Pat. No. 6,940,941 B2.

The gantry 1 is also fastened by means of a rotary joint 10 to a ceiling mount 8 (see FIG. 1 and FIG. 2) and can thus be positioned and moved in at least three degrees of freedom. It can also be moved by means of wheels on a ceiling rail 11. Alternatively the gantry can also be held on a multi-axis robot arm, for example a 6-axis articulated arm robot, allowing it to be moved in a flexible manner in the space.

Instead of the field emission emitter 2 it is also possible to use a conventional x-ray emitter 15, the x-ray radiation of which is converted by means of a coherence grating 14 to quasi-coherent x-ray radiation, as shown in FIG. 4. The x-ray detector apparatus can be formed for example by a flat solid state detector or a detector array curved along the gantry.

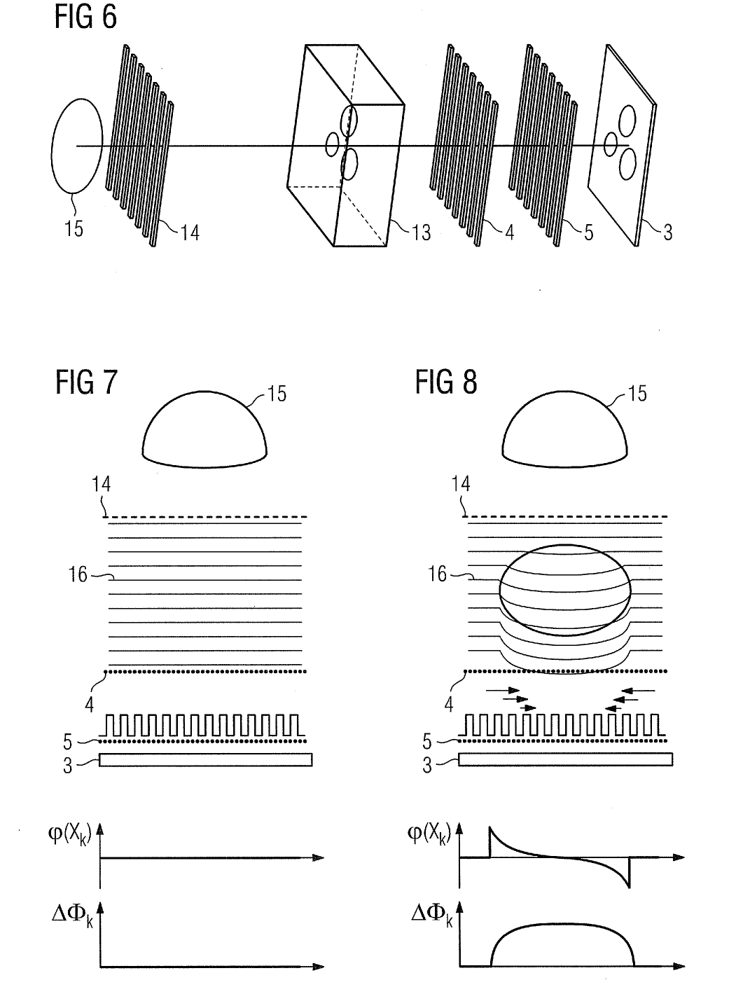

FIG. 6 shows a typical arrangement for phase contrast x-ray imaging with three aligned gratings, with one coherence grating 14 for producing coherent radiation from a non-coherent, conventional x-ray emitter 15, with the phase grating 4 for producing interference lines and with the amplitude grating 5 for reading out the interference pattern produced. The known radiation profile without and with examination object is shown in FIGS. 7 and 8. Coherent radiation 16 produced by means of the conventional x-ray emitter 15 and the coherence grating 14 strikes the phase grating 4, producing an interference pattern. The amplitude grating 5 disposed upstream of the x-ray detector 3 is used to read out the interference pattern. The amplitude grating 5 is moved and converts local interference lines to intensity fluctuations. The measured signal profile contains quantitative information about the phase gradients φ of the examination object 13.

Alternatively two or more recording systems may also be present in the gantry, with one recording system being configured for phase contrast x-ray imaging for example and the other for fluoroscopy imaging.

The invention can be summarized in brief as follows: to allow it to be deployed more effectively a computed tomography system is provided, which features an annular CT gantry with a central opening, a recording system that can be rotated within the gantry, having an x-ray source and an x-ray detector apparatus, it being possible to remove a part of the ring of the CT gantry from the ring at least partially, so that there is a break in the ring, through which an examination object can be moved into the central opening, the computed tomography system being configured for phase contrast x-ray imaging.

1.-12. (canceled)

13. A computed tomography system for phase contrast x-ray imaging, comprising:
an annular CT gantry having a ring segment with a central opening;
a hinge arranged on the ring segment; and
a recording system that rotates within the gantry having an x-ray source and an x-ray detector,
wherein a part of the ring segment is configured to be folded out by the hinge so that the ring segment is broken and through which an examination object is moved into the central opening.

14. The computed tomography system as claimed in claim 13, wherein the x-ray source comprises a plurality of field emission x-ray sources for emitting quasi-coherent x-ray radiation.

15. The computed tomography system as claimed in claim 14, wherein the field emission x-ray sources are disposed in an array.

16. The computed tomography system as claimed in claim 14, wherein each of the field emission x-ray sources comprises field emission cathodes with a nanostructured material and carbon nano tubes.

17. The computed tomography system as claimed in claim 13, further comprising a grating disposed behind the x-ray source for emitting coherent x-ray radiation from the x-ray source.

18. The computed tomography system as claimed in claim 13, further comprising:
a phase grating disposed between the examination object and the x-ray detector, and
an amplitude grating disposed between the phase grating and the x-ray detector.

19. The computed tomography system as claimed in claim 18, further comprising a device for moving the amplitude grating perpendicular to a radiation direction of an x-ray radiation emitted by the x-ray source.

20. The computed tomography system as claimed in claim 13, wherein the gantry comprises a first recording system and a second recording system each have one x-ray source and one x-ray detector.

21. The computed tomography system as claimed in claim 20, wherein the first recording system is configured for phase contrast x-ray imaging and the second recording system is configured for fluoroscopy x-ray imaging.

22. The computed tomography system as claimed in claim 13, wherein the CT gantry is pivotably disposed on a mount.

23. The computed tomography system as claimed in claim 22, wherein the mount is a ceiling mount.

24. The computed tomography system as claimed in claim 13, wherein the part of the ring segment is completely decoupled from the CT gantry.

25. The computed tomography system as claimed in claim 13, wherein the part of the ring segment is configured to pivot out from the ring segment.
